(19) United States
(12) Patent Application Publication
(10) Pub. No.: US 2025/0031678 A1
(43) Pub. Date: Jan. 30, 2025

(54) TRANSGENIC PURPLE BETTA AND METHODS OF MAKING THE SAME

(71) Applicant: GLOFISH, LLC, Earth City, MO (US)

(72) Inventor: Aidas NASEVICIUS, Tampa, FL (US)

(21) Appl. No.: 18/716,237

(22) PCT Filed: Nov. 21, 2022
(86) PCT No.: PCT/US2022/080223

§ 371 Date: Jun. 4, 2024

Related U.S. Application Data
(60) Provisional application No. 63/287,799, filed on Dec. 9, 2021.

(51) Int. Cl. A01K 67/0275

(52) U.S. Cl. CPC A01K 67/0275; A01K 2217/052; A01K 2227/40; A01K 2267/01

(57) ABSTRACT

The present disclosure relates to transgenic ornamental fish, as well as methods of making such fish by germ cell transplantation techniques. Also disclosed are methods of establishing a population of such transgenic fish and methods of providing them to the ornamental fish industry for the purpose of marketing, distributing and selling such transgenic fish.

### CROSS-REFERENCE TO RELATED APPLICATION

This application is being filed on Nov. 21, 2022, as a PCT International Patent Application and claims priority to and the benefit of U.S. Provisional Application No. 63/287,799, filed Dec. 9, 2021, which is incorporated by reference herein in its entirety.

### FIELD OF THE INVENTION

This invention relates to transgenic fish. Specifically, the invention relates to purple transgenic betta.

### INTRODUCTION

Transgenic technology involves the transfer of a foreign gene into a host organism enabling the host to acquire a new and inheritable trait. Transgenic technology has many potential applications. For example, it can be used to introduce a transgene into a fish in order to create new varieties of fish. There are many ways of introducing a foreign gene into fish, including: microinjection (e.g., Zhu et al., 1985; Du et al., 1992), electroporation (Powers et al., 1992), sperm-mediated gene transfer (Khoo et al., 1992; Sin et al., 1993), gene bombardment or gene gun (Zelenin et al., 1991), liposome-mediated gene transfer (Szelei et al., 1994), and the direct injection of DNA into muscle tissue (Xu et al., 1999). The first transgenic fish report was published by Zhu et al., (1985) using a chimeric gene construct consisting of a mouse metallothionein gene promoter and a human growth hormone gene. Most of the early transgenic fish studies have concentrated on growth hormone gene transfer with an aim of generating fast growing fish. While a majority of early attempts used heterologous growth hormone genes and promoters and failed to produce these fish (e.g. Chourrout et al., 1986; Penman et al., 1990; Brem et al., 1988; Gross et al., 1992), enhanced growth of transgenic fish has been demonstrated in several fish species including Atlantic salmon, several species of Pacific salmons, and loach (e.g. Du et al., 1992; Delvin et al., 1994, 1995; Tsai et al., 1995).

The Siamese fighting fish (Betta splendens), also sometimes colloquially known as the betta, is a species in the gourami family, which is popular as an aquarium fish. Species assigned to Betta splendens display a broad diversity of body shapes and coloration. The genus is well known among aquarists. Bettas can be territorial fish and are prone to high levels of aggression towards each other. Two males in proximity will almost always attack each other, if they do not have the ability to escape this will usually result in the death of one or both of the fish. Female bettas can also become territorial towards each other if they are housed in too small an aquarium.

Wild fish exhibit strong colors only when agitated. Breeders have been able to make this coloration permanent, and a wide variety of hues breed true. Colors available to the aquarist include red, orange, yellow, blue, steel blue, turquoise/green, black, pastel, white (“opaque” white, not to be confused with albino) and multi-colored fish.

Bettas are found in many different colors due to different layers of pigmentation in their skin. The layers (from furthest within to the outer layer) consists of red, yellow, black, iridescent (blue and green), and metallic (not a color of its own, but reacts with the other colors to change how they are perceived). Any combination of these layers can be present, leading to a wide variety of colors.

However, for the ornamental fish industry, the bold colors can be enhanced or novel colors can be introduced to provide a more efficient display of the various colors. The availability of such Bettas having modified pigmentation by transgenesis with fluorescent proteins would result in better products for the ornamental fish industry due to better visualization of the various colors.

Many fluorescent proteins are known in the art and have been used to investigate various cellular processes, including fluorescent proteins exhibiting various green, red, pink, yellow, orange, blue, or purple colors. Although transgenic experiments involving fluorescent proteins have provided new markers and reporters for transgenesis, progress in the field of developing and producing fish of various species that express such proteins has been limited and is unpredictable.

### Transgenic Purple Betta and Methods of Making the Same

In certain embodiments, the present disclosure concerns making transgenic fluorescent fish and providing such fish to the ornamental fish industry.

In some embodiments, transgenic fish or methods of making transgenic fish are provided. In certain aspects, the transgenic fish are fertile, transgenic, fluorescent fish. In a particular embodiment, the fish for use with the disclosed constructs and methods is the Betta. Betta skin color is determined by pigment cells in the skin, which contain pigment granules called melanosomes (black or brown color), xanthosomes (yellow color), erythrosomes (orange or red color), or iridosomes (iridescent colors, including white color). The number, size, and density of the pigment granules per pigment cell influence the color of the fish skin.

In certain specific embodiments, there are provided transgenic Betta or progeny thereof comprising specific transgenic integration events, referred to herein as transformation events. These fish are of particular interest because, for example, they embody an aesthetically pleasing purple color. Transgenic fish comprising these specific transgenic events may be homozygous or heterozygous (including, for example, hemizygous) for the transformation event. Homozygous fish bred with fish lacking a transformation event will in nearly all cases produce 100% heterozygous offspring. Germ cells, eggs, sperm, and embryos comprising these specific transgenic events are also included as part of the invention.

In one example embodiment regarding a specific transgenic integration event, a purple transgenic Betta or progeny thereof is provided comprising chromosomally integrated transgenes, wherein the Betta comprises the “Purple Betta 1 transformation event,” cryopreserved sperm cells comprising the Purple Betta 1 transformation event having been deposited at the American Type Culture Collections (ATCC), Historic District, 10801 University Blvd, Manassas, VA 20110. The chromosomally integrated transgenes may be present on one integrated expression cassette or two or more integrated expression cassettes. In certain aspects, such a transgenic Betta is a fertile, transgenic Betta. Such a transgenic Betta may be homozygous or heterozygous (including, for example, hemizygous) for the transgenes or integrated expression cassette(s).

Also disclosed are methods of providing a transgenic Betta comprising the Purple Betta 1 transformation event to the ornamental fish market. In some embodiments, the method comprises obtaining a transgenic Betta or progeny thereof comprising chromosomally integrated transgenes, wherein the Betta comprises the “Purple Betta 1 transformation event,” cryopreserved testes comprising the Purple Betta 1 transformation event being deposited at the ATCC, and distributing the fish to the ornamental fish market. Such fish may be distributed by a grower to a commercial distributor, or such fish may be distributed by a grower or a commercial distributor to a retailer such as, for example, a multi-product retailer having an ornamental fish department.

In some aspects, methods of producing a transgenic Betta are provided comprising: (a) obtaining a Betta that exhibits fluorescence and comprises one or more chromosomally integrated transgenes or expression cassettes, wherein the Betta comprises the “Purple Betta 1 transformation event,” cryopreserved sperm cells comprising the Purple Betta 1 transformation event being deposited at the ATCC; and (b) breeding the obtained Betta with a second Betta to provide a transgenic Betta comprising the Purple Betta 1 transformation event. The second Betta may be a transgenic or non-transgenic Betta.

In further embodiments, also provided are methods of producing a transgenic organism, the method comprising using cryopreserved sperm cells comprising the Purple Betta 1 transformation, such cryopreserved sperm cells deposited at the ATCC, to produce transgenic offspring. Such offspring may be, for example, a Betta, a species of the Betta splendens family, a fish species or genus related to Betta, or another fish species or genus. In some aspects, the fish may be produced using in vitro fertilization.

As used in this specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Any embodiment of any of the present methods, kits, and compositions may consist of or consist essentially of—rather than comprise/include/contain/have—the described features and/or steps. Thus, in any of the claims, the term “consisting of” or “consisting essentially of” may be substituted for any of the open-ended linking verbs recited above, in order to change the scope of a given claim from what it would otherwise be using the open-ended linking verb.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

### Transgenic Fish

In some aspects, the present disclosure regards transgenic fish. Methods of making transgenic fish are described in, for example, U.S. Pat. Nos. 7,135,613; 7,700,825; 7,834,239, each of which is incorporated by reference in its entirety. For example, a transgenic purple Betta may be generated using an expression cassette encoding purple fluorescent protein (PFP), such as FP635. Alternative purple fluorescent proteins include, but are not limited to Katushka2S, mKate2, mCherry2, mCherry, mKate-S158C, eqFP650, mPlum, jRed, and mRFPI.

It is preferred that fish belonging to species and varieties of fish of commercial value, particularly commercial value within the ornamental fish industry, be used. Such fish include but are not limited to bettas, catfish, zebrafish and other danios, medaka, carp, tilapia, goldfish, tetras, barbs, sharks (family cyprinidae, such as rainbow shark), angelfish, loach, koi, glassfish, discus, eel, goby, gourami, guppy, Xiphophorus, hatchet fish, Molly fish, or pangasius. A particular fish for use in the context of the present disclosure is a Betta. Bettas are increasingly popular ornamental animals and would be of added commercial value in various colors. Betta embryos are easily accessible and nearly transparent. Betta skin color is determined by pigment cells in the skin, which contain pigment granules called melanosomes. The number, size, and density of the melanosomes per pigment cell influence the color of the fish skin.

In commercial aquaculture, purple Bettas are spawned naturally pairwise. A mature male is placed into a 1-3 gallon tanks and left overnight. If the male builds a bubble nest, a female is added and left overnight. After the spawning, the female is removed and the male is left in the tank to take care of the developing eggs. After three days, the male is removed and the fry are fed freshly hatched Artemia nauplii for 3-4 weeks, until the fry are large enough to be safely moved to a grow-out vat. It takes about three months for bettas to become mature.

Line Regeneration from Cryopreserved Sperm Cells

Fish sperm freezing methods are well-known in the art; see, e.g., Walker and Streisinger (1983) and Draper and Moens (2007), both of which are incorporated herein by reference in their entireties. To obtain the transgenic fish disclosed herein, frozen betta sperm cells may be used to fertilize eggs.

In an example embodiment, young adult male purple bettas are euthanized using an overdose of sedative, such as Tricaine. The fish are decapitated, blotted dry, dissected and their testes are removed and transferred into ice-cold L-15 medium. Testes from approximately three fish per 100 μl of final sperm cell suspension should be collected. When the desired amount of tissue is obtained, the testes in L-15 medium are gently homogenized and centrifuged for approximately 2 seconds on a tabletop microfuge. The supernatant should be transferred into a new 1.7 ml tube taking care not to collect any pellet or fat from the surface. The collected suspension should be kept on ice. To the remaining pellet, add 100-300 μl of ice-cold L-15 medium and repeat the homogenization and centrifugation. Collect the supernatant as before and combine with the previously collected suspension. Repeat the addition of L-15 medium and homogenization three to five times or until the supernatant is not very cloudy; do not exceed the desired suspension volume. After collection, the sperm cell suspension should be frozen as soon as possible. It should be re-mixed immediately before freezing. For freezing, 15 μl of cryoprotectant solution (10% fat-free dry milk, 15% methanol, and 15% 2-methoxyethanol in L-15) should be transferred to a cryovial. 30 μl of sperm cell suspension should be added and mixed by pipetting 2-3 times. The cryovial should be closed, transferred into 15 ml centrifuge tube, and vertically embedded in dry ice. After 20 minutes, transfer the cryovials into liquid nitrogen. When convenient (when all the cryovials have been transferred or there is a long gap between the transfers) transfer the frozen cryotubes into a Dewar with liquid nitrogen for long term storage.

In example method of recovering a purple betta line, a mature male is placed into a 1-3 gallon tanks and left overnight. If the male builds a bubble nest, a female is added and left overnight. During the spawning, females should be collected. The bellies of the females should be blotted damp-dry with a paper towel. The eggs should not be exposed to water as this will prevent fertilization. The eggs should be squeezed out onto a slightly concave surface by applying light pressure to the sides of the posterior abdomen with a thumb and index finger and sliding the fingers forward to the genital pore. Eggs from several females may be pooled; the eggs can be kept unfertilized for several minutes. The sperm cells should be thawed at 30-33° C. in a water bath for 30 seconds. 70 p0 room temperature L-15 medium solution should be added to the vial and mixed. The sperm cell suspension is then immediately added to the eggs and gently mixed. The sperm and eggs are activated by adding 750 μl of fish water and mixing. The mixture should be incubated for 5 minutes at room temperature. The eggs then are transferred to small tanks where they are further cultured and reared to adulthood using regular protocol to obtain purple betta progeny; betta males should not be added to the developing embryos.

The present disclosure further encompasses progeny of a transgenic fish containing the Purple Betta 1 transformation event, as well as such transgenic fish derived from a transgenic fish egg, sperm cell, embryo, or other cell containing a genomically integrated transgenic construct. “Progeny,” as the term is used herein, can result from breeding two transgenic fish of the invention, or from breeding a first transgenic fish of the invention to a second fish that is not a transgenic fish of the invention. In the latter case, the second fish can, for example, be a wild-type fish, a specialized strain of fish, a mutant fish, or another transgenic fish. The second fish may be of the same species, or may be of a different species or genus. The hybrid progeny of these matings have the benefits of the transgene for fluorescence combined with the benefits derived from these other lineages.

The simplest way to identify fish containing the Purple Betta 1 transformation event is by visual inspection, as the fish in question would be purple colored and immediately distinguishable from non-transgenic fish.

In another example embodiment, fertile female purple bettas are bred with an adult male purple bettas. The fry are separated and categorized by color, for example, purple color compared to wild-type. Once the fry are categorized the purple bettas are allowed to develop and become fertile. At this stage, the purple betta fish may be used for additional genomic studies, used for next generation breeding or are euthanized. Euthanized purple bettas are obtained by using an overdose of sedative, such as Tricaine.

The fish are decapitated, blotted dry, dissected and their testes are removed and transferred into ice-cold L-15 medium. Testes from approximately three fish per 100 μl of final sperm cell suspension should be collected. When the desired amount of tissue is obtained, the testes in L-15 medium are gently homogenized and centrifuged for approximately 2 seconds on a tabletop microfuge. The supernatant should be transferred into a new 1.7 ml tube taking care not to collect any pellet or fat from the surface.

The collected suspension should be kept on ice. To the remaining pellet, add 100-300 μl of ice-cold L-15 medium and repeat the homogenization and centrifugation. Collect the supernatant as before and combine with the previously collected suspension. Repeat the addition of L-15 medium and homogenization three to five times or until the supernatant is not very cloudy; do not exceed the desired suspension volume. After collection, the sperm cell suspension should be frozen as soon as possible. It should be re-mixed immediately before freezing. For freezing, 15 μl of cryoprotectant solution (10% fat-free dry milk, 15% methanol, and 15% 2-methoxyethanol in L-15) should be transferred to a cryovial. 30 μl of sperm cell suspension should be added and mixed by pipetting 2-3 times. The cryovial should be closed, transferred into 15 ml centrifuge tube, and vertically embedded in dry ice. After 20 minutes, transfer the cryovials into liquid nitrogen. When convenient (when all the cryovials have been transferred or there is a long gap between the transfers) transfer the frozen cryotubes into a Dewar with liquid nitrogen for long term storage.

In example method of recovering a purple betta line, a mature male is placed into a 1-3 gallon tanks and left overnight. If the male builds a bubble nest, a female is added and left overnight. During the spawning, females should be collected. The bellies of the females should be blotted damp-dry with a paper towel. The eggs should not be exposed to water as this will prevent fertilization. The eggs should be squeezed out onto a slightly concave surface by applying light pressure to the sides of the posterior abdomen with a thumb and index finger and sliding the fingers forward to the genital pore. Eggs from several females may be pooled; the eggs can be kept unfertilized for several minutes. The sperm cells should be thawed at 30-33° C. in a water bath for 30 seconds. 70 p0 room temperature L-15 medium solution should be added to the vial and mixed. The sperm cell suspension is then immediately added to the eggs and gently mixed. The sperm and eggs are activated by adding 750 μl of fish water and mixing. The mixture should be incubated for 5 minutes at room temperature. The eggs then are transferred to small tanks where they are further cultured and reared to adulthood using regular protocol to obtain a second generation progeny purple betta The present disclosure further encompasses progeny of a transgenic fish containing the Purple Betta 1 transformation event, as well as such transgenic fish derived from a transgenic fish egg, sperm cell, embryo, or other cell containing a genomically integrated transgenic construct. “Progeny,” as the term is used herein, can result from breeding two transgenic fish of the invention, or from breeding a first transgenic fish of the invention to a second fish that is not a transgenic fish of the invention. In the latter case, the second fish can, for example, be a wild-type fish, a specialized strain of fish, a mutant fish, or another transgenic fish. The second fish may be of the same species, or may be of a different species or genus. The hybrid progeny of these matings have the benefits of the transgene for fluorescence combined with the benefits derived from these other lineages.

The simplest way to identify fish containing the Purple Betta 1 transformation event is by visual inspection, as the fish in question would be purple colored and immediately distinguishable from non-transgenic fish.

### Examples

Certain embodiments of the invention are further described with reference to the following examples. These examples are intended to be merely illustrative of the invention and are not intended to limit or restrict the scope of the present disclosure in any way and should not be construed as providing conditions, parameters, reagents, or starting materials that must be utilized exclusively in order to practice the art of the present disclosure.

### Example 1—Purple Transgenic Betta

Transgenic fish exhibiting a purple color are provided. The specific transgenic events embodied in these fish are designated the “Purple Betta 1 transformation event”. Sperm cells from these fish may be used to fertilize betta eggs and thereby breed transgenic betta that comprise these specific transgenic integration events. Cryopreserved sperm cells from this line were deposited at the ATCC, Historic District, 10801 University Blvd, Manassas, VA 20110, under the provisions of the Budapest Treaty as “Purple Betta 1”, under Provisional Accession Number PTA-127166.

### Example 2—Purple Transgenic Betta Progeny

Transgenic fish exhibiting a purple color are provided. The specific transgenic events embodied in these fish are designated the “Purple Betta 1 transformation event”. In some examples, the progeny of transgenic fish comprising the “Purple Betta 1 transformation event” also include at least a portion of the genetic sequence encoding the “Purple betta transformation event” such that the phenotype of the betta exhibits a purple physical appearance. Dissected cryopreserved testes from this progeny line may include at least 95%, 90%, 85% or 80% of the genetic sequence encoding the “Purple betta transformation event”. In at least these examples, the phenotype of the betta exhibits a purple physical appearance.

The fluorescent transgenic fish have use as ornamental fish in the market. Stably expressing transgenic lines can be developed by breeding a transgenic individual with a wild-type fish, mutant fish, or another transgenic fish. The desired transgenic fish can be distinguished from non-transgenic fish by observing the fish in white light, sunlight, ultraviolet light, blue light, or any other useful lighting condition that allows visualization of the purple color of the transgenic fish.

The fluorescent transgenic fish should also be valuable in the market for scientific research tools because they can be used for embryonic studies such as tracing cell lineage and cell migration. Additionally, these fish can be used to mark cells in genetic mosaic experiments and in fish cancer models.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. A transgenic Betta comprising a chromosomally integrated expression cassette encoding a fluorescent protein, wherein the Betta comprises the “Purple Betta 1 transformation event”.

2. The transgenic Betta of claim 1, wherein cryopreserved sperm cells comprising the Purple Betta 1 transformation event having been deposited at ATCC with Accession No. PTA-127166.

3. The transgenic Betta of claim 1, further defined as a fertile, transgenic Betta.

4. The transgenic Betta of claim 1, wherein the fish is homozygous for the integrated expression cassette.

5. The transgenic Betta of claim 1, wherein the fish is heterozygous for the integrated expression cassette.

6. A method of providing a transgenic Betta to the ornamental fish market, comprising obtaining a transgenic Betta in accordance with claim 1, and distributing the fish to the ornamental fish market.

7. The method of claim 6, wherein the fish are distributed by a grower to a commercial distributor.

8. The method of claim 6, wherein the fish are distributed by a grower or a commercial distributor to a retailer.

9. The method of claim 8, wherein the retailer is a multi-product retailer having an ornamental fish department.

10. A method of producing a transgenic Betta comprising:
(a) obtaining a Betta that comprises a chromosomally integrated expression cassette encoding a fluorescent protein, wherein the Betta comprises the “Purple Betta 1 transformation event”; and
(b) breeding the obtained Betta with a second Betta to provide a transgenic Betta comprising the Purple Betta 1 transformation event.

11. The method of claim 10, wherein cryopreserved sperm cells comprising the Purple Betta 1 transformation event having been deposited at ATCC with Accession No. PTA-127166

12. The method of claim 10, wherein the second Betta is a non-transgenic Betta.

13. A progeny of a transgenic Betta of claim 1 that comprises a chromosomally integrated expression cassette encoding a fluorescent protein, wherein the Betta and progeny exhibit fluorescence and comprise the “Purple Betta 1 transformation event,” cryopreserved sperm cells comprising the Purple Betta 1 transformation event having been deposited at ATCC.

14. The progeny fish of claim 13, further defined as a fertile, transgenic Betta.

15. The progeny fish of claim 13, wherein the fish is homozygous for the integrated expression cassette.

16. The progeny fish of claim 13, wherein the fish is heterozygous for the integrated expression cassette.

17. A method of providing a transgenic fish to the ornamental fish market, comprising obtaining a progeny fish in accordance with claim 13, and distributing the fish to the ornamental fish market.

18. The method of claim 17, wherein the fish are distributed by a grower to a commercial distributor.

19. The method of claim 18, wherein the fish are distributed by a grower or a commercial distributor to a retailer.

20. The method of claim 19, wherein the retailer is a multi-product retailer having an ornamental fish department.

21. A method of producing a transgenic fish comprising:
(a) obtaining a transgenic fish in accordance with claim 10; and
(b) breeding the obtained fish with a second fish to provide a transgenic fish comprising the Purple Betta 1 transformation event.

22. The method of claim 21, wherein the second fish is a non-transgenic fish.
